SEQUENCE LISTING (6 sequences)

SEQ ID NO: 1 - protein, 37 aa, Homo sapiens
DAEFRHDSGYEVHHQKLVFFAEDVGSNKGAIIGLMVG

SEQ ID NO: 2 - protein, 38 aa, Homo sapiens
DAEFRHDSGYEVHHQKLVFFAEDVGSNKGAIIGLMVGG

SEQ ID NO: 3 - protein, 40 aa, Homo sapiens
DAEFRHDSGYEVHHQKLVFFAEDVGSNKGAIIGLMVGGVV

SEQ ID NO: 4 - protein, 42 aa, Homo sapiens [sequence omitted]

SEQ ID NO: 5 - protein, 43 aa, Homo sapiens
DAEFRHDSGYEVHHQKLVFFAEDVGSNKGAIIGLMVGGVVIAT

SEQ ID NO: 6 - protein, 43 aa, Homo sapiens
VKMDAEFRHDSGYEVHHQKLVFFAEDVGSNKGAIIGLMVGGVV

(19) United States
(12) Patent Application Publication
(10) Pub. No.: US 2024/0159777 A1
(43) Pub. Date: May 16, 2024

(54) Biofluid-Based Methods for Diagnosing Alzheimer’s Disease-Associated Conditions

(71) Applicant: NeuroVision Imaging, Inc., Sacramento, CA (US)

(72) Inventors: Leyla Anderson, Elk Grove, CA (US); Hugo Vanderstichele, Gent (BE); Steven R. Verdooner, Granite Bay, CA (US)

(73) Assignee: NeuroVision Imaging, Inc., Sacramento, CA (US)

(21) Appl. No.: 18/550,321

(22) PCT Filed: Mar. 15, 2022
(86) PCT No.: PCT/US2022/020304

§ 371 Date: Sep. 13, 2023

Related U.S. Application Data
(60) Provisional application No. 63/161,524, filed on Mar. 16, 2021.

(51) Int. Cl. G01N 33/68; G01N 33/58

(52) U.S. Cl. CPC G01N 33/6896; G01N 33/58; G01N 33/6854; G01N 2333/4709; G01N 2333/4727; G01N 2800/2821

(57) ABSTRACT

This invention provides methods for determining whether a subject has a condition correlative with a matrix effect comprising (a) admixing (i) a suitably diluted sample of a suitable fluid from the subject and (ii) a suitable amount of a labeled molecule, wherein the labeled molecule is subject to a matrix effect with respect to the suitable fluid in a subject afflicted with the condition, and (b) after a suitable duration under suitable conditions, determining the amount of matrix-unaffected labeled molecule present in the resulting admixture, wherein the subject is afflicted with the condition if the amount of matrix-unaffected labeled molecule determined in step (b) correlates with a positive control for the condition, and wherein the subject is not afflicted with the condition if that amount correlates with a negative control for the condition. This invention also provides related methods, kits, and compositions. The present methods and kits are particularly useful for predicting the onset of neurodegenerative disorders such Alzheimer's disease and Parkinson's disease.

This application claims the benefit of U.S. Provisional Application No. 63/161,524, filed Mar. 16, 2021, the contents of which are incorporated herein by reference.

Throughout this application, various publications are cited. The disclosure of these publications is hereby incorporated by reference into this application to describe more fully the state of the art to which this invention pertains.

### FIELD OF THE INVENTION

The present invention relates to certain methods for determining whether a subject has a condition correlative with a matrix effect. These conditions can be diagnostic, prognostic, and/or correlative with disease progression for certain disorders, such as Alzheimer's disease.

### BACKGROUND OF THE INVENTION

Alzheimer's disease is characterized as a continuous change in severity of different pathologies, such as β-amyloid (Aβ) accumulation in the brain. Other Alzheimer's-related pathologies include, for example, tauopathy and synapse loss. Diagnostic and prognostic methods for Alzheimer's disease based on measuring different plasma Aβ peptides or (phospho-) taus have been explored. However, these methods have yielded contradictory results, and distinctions in absolute values between healthy control subjects and diseased subjects are limited. Thus, there remains a need for accurate biofluid-based methods for diagnosing Alzheimer's disease, predicting the onset of Alzheimer's disease, and evaluating Alzheimer's disease progression.

### SUMMARY OF THE INVENTION

This invention provides a first method for determining whether a subject has a condition correlative with a matrix effect comprising (a) admixing (i) a suitably diluted sample of a suitable fluid from the subject and (ii) a suitable amount of a labeled molecule, wherein the labeled molecule is subject to a matrix effect with respect to the suitable fluid in a subject afflicted with the condition, and (b) after a suitable duration under suitable conditions, determining the amount of matrix-unaffected labeled molecule present in the resulting admixture, wherein the subject is afflicted with the condition if the amount of matrix-unaffected labeled molecule determined in step (b) correlates with a positive control for the condition, and wherein the subject is not afflicted with the condition if that amount correlates with a negative control for the condition.

This invention also provides a second method for determining whether a subject has a condition correlative with a matrix effect comprising (a) admixing (i) a suitably diluted sample of a suitable fluid from the subject and (ii) suitable amounts of a first labeled molecule and a second labeled molecule, wherein the labeled molecules are subject to a matrix effect with respect to the suitable fluid in a subject afflicted with the condition, and (b) after a suitable duration under suitable conditions, determining the quantitative relationship between matrix-unaffected first labeled molecule and matrix-unaffected second labeled molecule present in the resulting admixture, wherein the subject is afflicted with the condition if the relationship determined in step (b) correlates with a positive control for the condition, and wherein the subject is not afflicted with the condition if that relationship correlates with a negative control for the condition.

This invention further provides a first kit for use in determining whether a subject (preferably a human subject) has a condition correlative with a matrix effect comprising, in separate compartments, (a) a labeled molecule, wherein the labeled molecule is subject to a matrix effect with respect to a suitable fluid in a subject afflicted with the condition, and (b) an agent useful for treating the labeled molecule to facilitate its measurement in matrix-unaffected form. This invention still further provides a second kit for use in determining whether a subject (preferably a human subject) has a condition correlative with a matrix effect comprising, in separate compartments, (a) a first labeled molecule and a second labeled molecule, wherein each labeled molecule is subject to a matrix effect with respect to a suitable fluid in a subject afflicted with the condition, and (b) an agent useful for treating each of the first and second labeled molecules to facilitate its measurement in matrix-unaffected form.

This invention provides a first method for determining whether a human subject (either symptomatic or asymptomatic) has a condition correlative with Alzheimer's disease (i.e., wherein the subject may or may not actually be afflicted yet with Alzheimer's disease) comprising (a) admixing (i) a suitably diluted sample of a suitable fluid from the subject and (ii) a suitable amount of a labeled Aβ peptide, and (b) after a suitable duration under suitable conditions, determining the amount of matrix-unaffected labeled Aβ peptide present in the resulting admixture, wherein the subject is afflicted with the condition if the amount of matrix-unaffected labeled Aβ peptide determined in step (b) correlates with a positive control for the condition, and wherein the subject is not afflicted with the condition if that amount correlates with a negative control for the condition.

This invention also provides a second method for determining whether a human subject (either symptomatic or asymptomatic) has a condition correlative with Alzheimer's disease (i.e., wherein the subject may or may not actually be afflicted yet with Alzheimer's disease) comprising (a) admixing (i) a suitably diluted sample of a suitable fluid from the subject and (ii) suitable amounts of a first labeled Aβ peptide and a second labeled Aβ peptide, and (b) after a suitable duration under suitable conditions, determining the quantitative relationship between matrix-unaffected first labeled Aβ peptide and matrix-unaffected second labeled Aβ peptide present in the resulting admixture, wherein the subject is afflicted with the condition if the relationship determined in step (b) correlates with a positive control for the condition, and wherein the subject is not afflicted with the condition if that relationship correlates with a negative control for the condition.

This invention further provides a first kit for use in determining whether a human subject has a condition correlative with Alzheimer's disease comprising, in separate compartments, (a) a labeled Aβ peptide, and (b) an agent useful for treating the labeled Aβ peptide to facilitate the labeled Aβ peptide's measurement in its matrix-unaffected form.

This invention still further provides a second kit for use in determining whether a human subject has a condition correlative with Alzheimer's disease comprising, in separate compartments, (a) a first labeled Aβ peptide and a second labeled Aβ peptide, and (b) an agent useful for treating each of the first and second labeled Aβ peptides to facilitate the labeled Aβ peptide's measurement in its matrix-unaffected form.

This invention provides a first method for determining whether a human subject (either symptomatic or asymptomatic) has a condition correlative with Parkinson's disease (i.e., wherein the subject may or may not actually be afflicted yet with Parkinson's disease) comprising (a) admixing (i) a suitably diluted sample of a suitable fluid from the subject and (ii) a suitable amount of a labeled α-synuclein, and (b) after a suitable duration under suitable conditions, determining the amount of matrix-unaffected labeled α-synuclein present in the resulting admixture, wherein the subject is afflicted with the condition if the amount of matrix-unaffected labeled α-synuclein determined in step (b) correlates with a positive control for the condition, and wherein the subject is not afflicted with the condition if that amount correlates with a negative control for the condition.

This invention also provides a second method for determining whether a human subject (either symptomatic or asymptomatic) has a condition correlative with Parkinson's disease (i.e., wherein the subject may or may not actually be afflicted yet with Parkinson's disease) comprising (a) admixing (i) a suitably diluted sample of a suitable fluid from the subject and (ii) suitable amounts of a first labeled α-synuclein and a second labeled α-synuclein, and (b) after a suitable duration under suitable conditions, determining the quantitative relationship between matrix-unaffected first labeled α-synuclein and matrix-unaffected second labeled α-synuclein present in the resulting admixture, wherein the subject is afflicted with the condition if the relationship determined in step (b) correlates with a positive control for the condition, and wherein the subject is not afflicted with the condition if that relationship correlates with a negative control for the condition.

This invention further provides a first kit for use in determining whether a human subject has a condition correlative with Parkinson's disease comprising, in separate compartments, (a) a labeled α-synuclein, and (b) an agent useful for treating the labeled α-synuclein to facilitate the labeled α-synuclein's measurement in its matrix-unaffected form.

Finally, this invention provides a second kit for use in determining whether a human subject has a condition correlative with Parkinson's disease comprising, in separate compartments, (a) a first labeled α-synuclein and a second labeled α-synuclein, and (b) an agent useful for treating each of the first and second labeled α-synucleins to facilitate the labeled α-synucleins' measurement in their matrix-unaffected form.

### BRIEF DESCRIPTION OF THE FIGURES

FIG. 1 This figure shows results from Assay 1 described in Example 2 below. Results are presented as box-plots for each study. Details on the outcome parameters are described in Table 1.

FIG. 2 This figure shows results from Assay 2 described in Example 3 below. Results are expressed as mean±standard error (n=8).

FIG. 3 This figure shows results from Assay 3 described in Example 4 below. Results are presented as parts per million (ppm) values for one sample at various dilutions of the sample.

### DETAILED DESCRIPTION OF THE INVENTION

This invention provides certain methods for determining whether a subject has a condition correlative with a matrix effect. These matrix-related correlations can be diagnostic and/or prognostic for certain disorders, particularly neurodegenerative disorders.

### Definitions

In this application, certain terms are used which shall have the meanings set forth as follows.

As used herein, an “Aβ peptide” includes, without limitation, the peptides having the amino acid sequences set forth in Example 5. An Aβ peptide also includes, without limitation, a fragment of any of Aβ1-37 peptide, Aβ1-38 peptide, Aβ1-40 peptide, Aβ1-42 peptide, Aβ1-43 peptide, Aβ1-10 peptide, Aβ1-16 peptide, Aβ(-3)-37 peptide, Aβ(-3)-38 peptide, Aβ(-3)-40 peptide, Aβ(-3)-42 peptide, Aβ(-3)-43 peptide, Aβ(-3)-10 peptide, or Aβ(-3)-16 peptide. These fragments include, for example, (i) N-terminal truncated fragments of Aβ1-37 peptide, Aβ1-38 peptide, Aβ1-40 peptide, Aβ1-42 peptide, Aβ1-43 peptide, Aβ1-10 peptide, and Aβ1-16 peptide; (ii) C-terminal truncated fragments ending at position Aβ37, Aβ38, Aβ40, Aβ42, Aβ43, Aβ10, and Aβ16; and (iii) fragments of Aβ37 peptide, Aβ38 peptide, Aβ40 peptide, Aβ42 peptide, Aβ43 peptide, Aβ10 peptide, and Aβ16 peptide that are truncated or elongated at both the N- and C-termini.

Aβ peptide fragments contemplated herein include, for example, (i) an Aβ37 peptide fragment having a length (in amino acid residues) of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36; (ii) an Aβ38 peptide fragment having a length (in amino acid residues) of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or 37; (iii) an Aβ40 peptide fragment having a length (in amino acid residues) of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39; (iv) an Aβ42 peptide fragment having a length (in amino acid residues) of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or 41; (v) an Aβ43 peptide fragment having a length (in amino acid residues) of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42; (vi) an Aβ10 peptide fragment having a length (in amino acid residues) of 5, 6, 7, 8, or 9; and (vii) an Aβ16 peptide fragment having a length (in amino acid residues) of 5, 6, 7, 8, 9, 10, 11, 12, 13, 14, or 15.

Aβ peptide fragments contemplated herein also include, for example, the following, wherein the fragment's N-terminus is defined as the corresponding amino acid residue number of its full-length counterpart Aβ peptide: (i) an Aβ37 peptide fragment having its N-terminus at residue 2, 3, 4, 5, 6, 7, 8, 9, 10, 11, 12, 13, 14, 15, 16, 17, 18, 19, 20, 21, 22, 23, 24, 25, 26, or 27; (ii) an Aβ38 peptide fragment having its N-terminus at residue 2, 3, 4, 5, 6, 7, 8, 9, 10, 11, 12, 13, 14, 15, 16, 17, 18, 19, 20, 21, 22, 23, 24, 25, 26, 27, or 28; (iii) an Aβ40 peptide fragment having its N-terminus at residue 2, 3, 4, 5, 6, 7, 8, 9, 10, 11, 12, 13, 14, 15, 16, 17, 18, 19, 20, 21, 22, 23, 24, 25, 26, 27, 28, 29, 30; (iv) an Aβ42 peptide fragment having its N-terminus at residue 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or 32; (v) an Aβ43 peptide fragment having its N-terminus at residue 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33; (vi) an Aβ10 peptide fragment having its N-terminus at residue 2, 3, 4, or 5; and (vii) an Aβ16 peptide fragment having its N-terminus at residue 2, 3, 4, 5, or 6. Aβ peptides and fragments thereof may optionally contain amino acid derivatives, such as pyroglutamic acid (e.g., a pyroglutamic acid substitution at amino acid residues 3 and/or 11 (Gu and Viles (2016), and Rezaei-Ghaleh, et al. (2016)).

As used herein, a “condition correlative with a matrix effect” includes, without limitation, an amyloidopathy, a tauopathy, neuronal damage, a synucleinopathy, a cerebral proteinopathy (e.g., TDP-43 proteinopathy), a progranulinopathy, and neuronal inflammation. In this invention, the following conditions, without limitation, are envisioned together with the following proteins and peptides that are subject to a matrix effect and, when labeled, are useful for performing the present methods. (i) Amyloidopathy (β-amyloid; BACE1 (beta-site amyloid precursor protein cleaving enzyme); and APP (β-amyloid precursor protein)). (ii) Tauopathy (tau and phosphorylated tau). (iii) Synucleopathy (α-synuclein; neurogranin; SNAP-25 (synaptosome-associated protein 25); GAP-43 (growth-associated protein 43 or neuromodulin); synaptotagmin; VAMP2 (vesicle-associated membrane protein 2) and synpatobrevin). (iv) TDP-43 pathology (TDP-43 (transactive response DNA binding protein)). (v) Neuroinflammation and glial activation (sTREM2 B36 (soluble triggering receptor expressed on myeloid cells 2); YKL-(chitinase-3-like protein-1); IP-10 (interferon-gamma-induced protein 10); GFAP (glial fibrillary acidic protein); progranulin; osteopontin; MCP-1 (monocyte chemotactic protein-1); and IL-6 (Interleukin-6)). (vi) Vascular dysregulation (hFABP (heart-type fatty-acid-binding protein)). (vii) Neuronal injury (VILIP-1 (Visinin-Like Protein 1); and neurofilament).

As used herein, the term “antibody” includes, without limitation, (a) an immunoglobulin molecule comprising two heavy chains (i.e., H chains, such as μ, δ, γ, α and ε) and two light chains (i.e., L chains, such as λ and κ) and which recognizes an antigen; (b) polyclonal and monoclonal immunoglobulin molecules; (c) monovalent and divalent fragments thereof, and (d) bispecific forms thereof. Immunoglobulin molecules may derive from any of the commonly known classes, including but not limited to IgA, secretory IgA, IgG and IgM. IgG subclasses are also well known to those in the art and include, but are not limited to, human IgG1, IgG2, IgG3 and IgG4. Antibodies can be both naturally occurring and non-naturally occurring. Furthermore, antibodies include chimeric antibodies, wholly synthetic antibodies, single chain antibodies (e.g., scFv), and fragments thereof. Antibodies may contain, for example, all or a portion of a constant region (e.g., an Fc region) and a variable region, or contain only a variable region (responsible for antigen binding). Antibodies may be human, humanized, or nonhuman (e.g., camel) antibodies.

As used herein, a “human subject” can be of any age, gender, race, or state of co-morbidity. In one embodiment, the subject is male, and in another, the subject is female. In another embodiment, the subject is younger than 60 years old, younger than 55 years old, younger than 50 years old, younger than 45 years old, younger than 40 years old, younger than 35 years old, younger than years old, younger than 25 years old, or younger than 20 years old. In another embodiment, the subject carries a genetic mutation (e.g., a mutation in APP) correlative with the early onset of Alzheimer's disease. In yet another embodiment, the subject is at least 60 years old, at least 65 years old, at least 70 years old, at least 75 years old, at least 80 years old, at least 85 years old, or at least 90 years old. In a further embodiment, the human subject is symptomatic of a disorder (e.g., Alzheimer's disease or Parkinson's disease) associated with the condition detected using the present methods. In yet a further embodiment, the human subject is asymptomatic of a disorder (e.g., Alzheimer's disease or Parkinson's disease) associated with the condition detected using the present methods.

As used herein, “labeled”, with respect to a molecule, means covalently or non-covalently affixed to a detectable moiety. In one embodiment, the detectable moiety is directly affixed to the molecule (i.e., the detectable moiety is bound to the molecule without being bound via an intermediary moiety). In another embodiment, the detectable moiety is indirectly affixed to the molecule (i.e., the detectable moiety is bound to the molecule via an intermediary moiety such as a polypeptide or other type of linker). Detectable moieties include, for example, radioisotopes (e.g., ³²P, ³⁵S, and ¹²⁵I), fluorophores, and other non-radioactive compounds (e.g., biotin). When the labeled molecule is a peptide or protein, for example, the label can be affixed to the N-terminus, to the C-terminus, to a central domain, or to some or all of the peptide's or protein's amino acid residues. Labeled peptides and proteins include, for example, labeled β-amyloid, labeled tau, labeled synuclein, and labeled TDP-43. For example, where the labeled peptide is biotin-labeled Aβ40 peptide, the biotin can be bound to the N-terminus, to the C-terminus, or to a central domain. Additional examples of biotin-labeled peptides include the following: (i) N-terminally labeled Aβ37 peptide, C-terminally labeled Aβ37 peptide, and centrally labeled Aβ37 peptide; (ii) N-terminally labeled Aβ38 peptide, C-terminally labeled Aβ38 peptide, and centrally labeled Aβ38 peptide; (iii) N-terminally labeled Aβ42 peptide, C-terminally labeled Aβ42 peptide, and centrally labeled Aβ42 peptide; (iv) N-terminally labeled Aβ43 peptide, C-terminally labeled Aβ43 peptide, and centrally labeled Aβ43 peptide; (v) N-terminally labeled Aβ10 peptide, C-terminally labeled Aβ10 peptide, and centrally labeled Aβ10 peptide; (vi) N-terminally labeled α-synuclein, C-terminally labeled α-synuclein, and centrally labeled α-synuclein; (vii) N-terminally labeled tau protein, C-terminally labeled tau protein, and centrally labeled tau protein; (viii) N-terminally labeled TDP-43, C-terminally labeled TDP-43, and centrally labeled TDP-43; (ix) N-terminally labeled progranulin, C-terminally labeled progranulin, and centrally labeled progranulin; (x) N-terminally labeled GFAP, C-terminally labeled GFAP, and centrally labeled GFAP; (xi) N-terminally labeled neurogranin, C-terminally labeled neurogranin, and centrally labeled neurogranin; (xii) N-terminally labeled neurofilament, C-terminally labeled neurofilament, and centrally labeled neurofilament; and (xiii) N-terminally labeled phosphorylated tau protein, C-terminally labeled phosphorylated tau protein, and centrally labeled phosphorylated tau protein. Also envisioned in this invention are N-terminally, C-terminally, and centrally labeled fragments of each labeled peptide and protein set forth in items (i)-(xiii) above. Preferably, for a labeled molecule (e.g., a biotin-labeled Aβ peptide), the label is bound to the molecule in a manner that does not interfere with (i) the labeled molecule's measurement, or (ii) binding to a capture antibody. The ideal manner for labeling a molecule to achieve that end depends on the molecule, the label, and the measurement method used. So, for example, when the molecule is Aβ40 peptide, the label is biotin, and the measurement method used employs an anti-biotin antibody, avidin, or streptavidin (e.g., radiolabeled, enzymatically labeled, or magnetic particle-bound streptavidin), the ideal manner for labeling the Aβ40 peptide with biotin might be to bind biotin to the peptide's N-terminus.

As used herein, a “matrix effect” is a phenomenon whereby, with respect to a certain condition (e.g., an amyloidopathy) in a subject, a suitably diluted sample of a suitable fluid from the subject (e.g., a 32-fold diluted plasma sample), when admixed with a suitable amount of a labeled molecule (e.g., 100 pg/ml of a biotin-labeled Aβ peptide) for a suitable time under suitable conditions (e.g., for three hours at room temperature), yields a greater amount of matrix-unaffected labeled molecule (i.e., a lower amount of labeled molecule bound to biomolecules (e.g., carrier proteins) in the fluid) when the fluid is from a subject having the condition than from a subject not having the condition. For example, a matrix effect is demonstrated when, for a subject having an amyloidopathy, a 32-fold diluted plasma sample from the subject is admixed with 100 pg/ml of biotin-labeled Aβ40 peptide for three hours at room temperature and yields a greater amount of matrix-unaffected biotin-labeled Aβ40 peptide than would a plasma sample from a subject not having an amyloidopathy. Without wishing to be bound by any particular scientific theory, it is believed that available biomolecules like carrier proteins (e.g., chaperone proteins) in the diluted fluid sample act as a “matrix” with respect to the labeled molecule, in that this matrix binds the labeled molecule to an extent that correlates with the presence or absence of the condition being detected. Specifically, when the condition is present, the matrix may have a different amount of available binding proteins, binding proteins with a modified three-dimensional structure, binding proteins with secondary modifications (e.g., phosphorylation, nitration, and glycation), or a decreased capacity to carry, and thus binds less of the labeled molecule than it could when the condition is absent. Therefore, when the condition is present, more matrix-unaffected labeled molecules result than when the condition is absent.

As used herein, a “matrix-unaffected” labeled molecule means a labeled molecule that is not adsorbed or otherwise bound to any biomolecules (e.g., carrier proteins) from a subject's fluid sample that would interfere with its detection.

As used herein, the term “molecule” includes, without limitation, an aptamer, a polypeptide (e.g., a peptide (such as a synthetic peptide) or a protein (such as a recombinant protein)), a nucleic acid (e.g., DNA molecule or an RNA molecule), a lipoprotein, and a carbohydrate.

As used herein, a “negative control”, with respect to the present methods, includes, without limitation, (i) the amount of matrix-unaffected labeled molecule (or first and second matrix-unaffected labeled molecules, as applicable) determined in a concurrently performed parallel method using fluid from a subject known not to have the condition being tested for; (ii) the amount of matrix-unaffected labeled molecule (or first and second matrix-unaffected labeled molecules, as applicable) determined in a previously performed parallel method using fluid from a subject known not to have the condition being tested for; (iii) the mean or median amount (±15%) of matrix-unaffected labeled molecule (or first and second matrix-unaffected labeled molecules, as applicable) determined in a plurality of previously performed parallel methods using fluid from one or more subjects known not to have the condition being tested for; and (iv) the mean or median relationship (e.g., mean ratio) between first and second matrix-unaffected labeled molecules determined in a plurality of previously performed parallel methods using fluid from one or more subjects known not to have the condition being tested for. By way of example, in one embodiment, the present method is for determining whether a human subject has an amyloidopathy correlative with Alzheimer's disease. The method comprises (a) admixing (i) a 32-fold diluted sample of plasma from the subject and (ii) 100 pg/ml of biotin-labeled Aβ40 peptide, and (b) after a three hours at room temperature, determining the amount of matrix-unaffected biotin-labeled Aβ40 peptide present in the resulting admixture. Assume that the negative control is 100 ppm, which is the mean amount of matrix-unaffected biotin labeled Aβ40 peptide determined in 20 previously performed parallel methods (i.e., performed identically to the present method except that they use 32-fold diluted plasma from 20 subjects known not to have the amyloidopathy correlative with Alzheimer's disease). Also assume that the amount of matrix-unaffected biotin-labeled Aβ40 peptide present in the resulting admixture is determined to be 100 ppm. The subject does not have the amyloidopathy because the amount of matrix-unaffected labeled Aβ peptide determined in step (b) correlates with the negative control for the amyloidopathy. In this invention, a perfect correlation with the negative control is not necessary to determine that the subject does not have the condition being tested for. For instance, in the example above, one could still determine that the subject does not have the amyloidopathy if the amount of matrix-unaffected biotin-labeled Aβ40 peptide present in the admixture were determined to be 125 ppm, the negative control were 100 ppm, and the positive control (discussed herein) were 300 ppm.

As used herein, a “neurodegenerative disorder” includes, without limitation, Alzheimer's disease and Parkinson's disease.

As used herein, a “plurality of serially diluted samples” (e.g., plasma samples) includes, without limitation, two or more samples of differing dilution factor. In one embodiment, a plurality of serially diluted samples comprises two samples wherein the fluid therein is diluted by (i) a factor of 4 and a factor of 8; (ii) a factor of 8 and a factor of 16; (iii) a factor of 16 and a factor of 32; (iv) a factor of 32 and a factor of 64; (v) a factor of 64 and a factor of 128; and (vi) a factor of 128 and a factor of 256. In another embodiment, a plurality of serially diluted samples comprises three samples wherein the fluid therein is diluted by (i) a factor of 4, a factor of 8, and a factor of 16; (ii) a factor of 8, a factor of 16, and a factor of 32; (iii) a factor of 16, a factor of 32, and a factor of 64; (iv) a factor of 32, a factor of 64, and a factor of 128; and (v) a factor of 64, a factor of 128, and a factor of 256. In a further embodiment, a plurality of serially diluted samples comprises four samples wherein the fluid therein is diluted by (i) a factor of 4, a factor of 8, a factor of 16, and a factor of 32; (ii) a factor of 8, a factor of 16, a factor of 32, and a factor of 64; (iii) a factor of 16, a factor of 32, a factor of 64, and a factor of 128; and (iv) a factor of 32, a factor of 64, a factor of 128, and a factor of 256. In yet a further embodiment, a plurality of serially diluted samples comprises five samples wherein the fluid therein is diluted by (i) a factor of 4, a factor of 8, a factor of 16, a factor of 32, and a factor of 64; (ii) a factor of 8, a factor of 16, a factor of 32, a factor of 64, and a factor of 128; and (iii) a factor of 16, a factor of 32, a factor of 64, a factor of 128, and a factor of 256. In yet a further embodiment, a plurality of serially diluted samples comprises six samples wherein the fluid therein is diluted by (i) a factor of 4, a factor of 8, a factor of 16, a factor of 32, a factor of 64, and a factor of 128; and (ii) a factor of 8, a factor of 16, a factor of 32, a factor of 64, a factor of 128, and a factor of 256. In still a further embodiment, a plurality of serially diluted samples comprises seven samples wherein the fluid therein is diluted by a factor of 4, a factor of 8, a factor of 16, a factor of 32, a factor of 64, a factor of 128, and a factor of 256.

As used herein, a “positive control”, with respect to the present methods, includes, without limitation, (i) the amount of matrix-unaffected labeled molecule (or first and second matrix-unaffected labeled molecules, as applicable) determined in a concurrently performed parallel method using fluid from a subject known to have the condition being tested for; (ii) the amount of matrix-unaffected labeled molecule (or first and second matrix-unaffected labeled molecules, as applicable) determined in a previously performed parallel method using fluid from a subject known to have the condition being tested for; (iii) the mean or median amount of matrix-unaffected labeled molecule (or first and second matrix-unaffected labeled molecules, as applicable) determined in a plurality of previously performed parallel methods using fluid from one or more subjects known to have the condition being tested for; and (iv) the mean or median relationship (e.g., mean ratio) between first and second matrix-unaffected labeled molecules determined in a plurality of previously performed parallel methods using fluid from one or more subjects known to have the condition being tested for. By way of example, in one embodiment, the present method is for determining whether a human subject has an amyloidopathy correlative with Alzheimer's disease. The method comprises (a) admixing (i) a 32-fold diluted sample of plasma from the subject and (ii) 100 pg/ml of biotin-labeled Aβ40 peptide, and (b) after a three hours at room temperature, determining the amount of matrix-unaffected biotin-labeled Aβ40 peptide present in the resulting admixture. Assume that the positive control is 300 ppm, which is the mean amount of matrix-unaffected biotin labeled Aβ40 peptide determined in 20 previously performed parallel methods (i.e., performed identically to the present method except that they use 32-fold diluted plasma from 20 subjects known to have the amyloidopathy correlative with Alzheimer's disease). Also assume that the amount of matrix-unaffected biotin-labeled Aβ40 peptide present in the resulting admixture is determined to be 300 ppm. The subject has the amyloidopathy because the amount of matrix-unaffected labeled Aβ peptide determined in step (b) correlates with the positive control for the amyloidopathy. In this invention, a perfect correlation with the positive control is not necessary to determine that the subject has the condition being tested for. For instance, in the example above, one could still determine that the subject has the amyloidopathy if the amount of matrix-unaffected biotin-labeled Aβ40 peptide present in the admixture were determined to be 275 ppm, the positive control were 300 ppm, and the negative control (discussed herein) were 100 ppm.

As used herein, a “quantitative relationship” between a matrix-unaffected first labeled molecule and a matrix-unaffected second labeled molecule present in a resulting admixture includes, without limitation, (i) a change in one of the molecules relative to the other; (ii) the ratio of the amount of one of the two molecules to that of the other (e.g., the ratio of the matrix-unaffected first labeled molecule to the matrix-unaffected second labeled molecule); (iii) a relationship defined by an algorithm (e.g., x*A+b*B+z); (iv) the relationship between the slopes of each molecule's dilution curve; (v) the relative end dilution titers of each molecule; and (vi) combinations thereof.

As used herein, an antibody “specifically binds” to its target (e.g., an epitope on an Aβ peptide) if it does at least one of the following: (i) binds to its target with an affinity greater than that with which it binds to any other target; or (ii) binds to its target with an affinity (i.e., an equilibrium dissociation constant) of at least 5×10⁻⁴ M (e.g., at least 1×10⁻⁴ M, at least 5×10⁻⁵ M, at least 1×10⁻⁵ M, at least 5×10⁻⁶ M, at least 1×10⁻⁶ M, at least 5×10⁻⁷ M, at least 1×10⁻⁷ M, at least 5×10⁻⁸ M, at least 1×10⁻⁸ M, at least 5×10⁻⁹ M, or at least 1×10⁻⁹ M). Preferably, an antibody specifically binds to its target if it performs both of items (i) and (ii) above.

As used herein, the term “subject” includes, without limitation, a mammal such as a human, a non-human primate, a dog, a cat, a horse, a sheep, a goat, a cow, a rabbit, a pig, a hamster, a rat and a mouse. The present methods are envisioned for these non-human embodiments, mutatis mutandis, as they are for human subjects in this invention.

As used herein, a “suitable amount” of a labeled molecule is an amount sufficient to permit experimental detection of the molecule in matrix-unaffected form in step (b) of the present methods. Suitable amounts of a labeled molecule include, without limitation, (i) 1 pg/ml (i.e., an amount of labeled molecule to achieve a concentration of 1 pg/ml), 5 pg/ml, 10 pg/ml, 20 pg/ml, pg/ml, 40 pg/ml, 50 pg/ml, 60 pg/ml, 70 pg/ml, 80 pg/ml, 90 pg/ml, 100 pg/m, 110 pg/m, 120 pg/m, 130 pg/m, 140 pg/m, 150 pg/m, 160 pg/m, 170 pg/m, 180 pg/m, 190 pg/m, 200 pg/m, 300 pg/m, 400 pg/m, 500 pg/m, 600 pg/ml, 700 pg/ml, 800 pg/ml, 900 pg/ml, 1 ng/ml, 5 ng/ml, 10 ng/ml, 50 ng/ml, and 100 ng/ml; (ii) from 1 pg/ml to 20 pg/ml, from 20 pg/ml to 50 pg/ml, from 50 pg/ml to 100 pg/ml, from 100 pg/ml to 150 pg/ml, from 150 pg/ml to 200 pg/ml, from 200 pg/ml to 500 pg/ml, from 500 pg/ml to 1 ng/ml, from 1 ng/ml to 10 ng/ml, and from 10 ng/ml to 100 ng/ml; and (iii) from 1 pg/ml to 100 pg/ml, from 100 pg/ml to 1 ng/ml, from 1 ng/ml to 10 ng/ml, and from 10 ng/ml to 100 ng/ml.

As used herein, a “suitable fluid” includes, without limitation, whole blood, serum, plasma (e.g., untreated plasma, and plasma treated with EDTA, heparin, and/or citrate), cerebrospinal fluid (e.g., lumbar, ventricular, or cisternal CSF, whether collected via shunt or drain), vitreous humor, saliva, tear fluid, ocular fluid, nasal fluid, and interstitial fluid.

As used herein, “suitable conditions”, with respect to step (b) of the present methods, includes, without limitation, (i) room temperature, 37° C., and/or 2-8° C.; (ii) rotation, orbital shaking and/or stationary; (iii) fresh, frozen (different cycles/methods) (e.g., wherein the temperature decreases to freezing), heated (i.e., wherein the temperature increases to 56° C. or higher), and/or unaltered; (iv) sonication; (v) denaturation (e.g., via GuHCl, formic acid, high concentrations of detergents, and combinations thereof); and (vi) combinations thereof.

As used herein, a “suitable duration”, with respect to step (b) of the present methods, includes, without limitation, 5 minutes, 10 minutes, 20 minutes, 30 minutes, 40 minutes, 50 minutes, 60 minutes, 1.5 hours, 2 hours, 2.5 hours, 3 hours, 3.5 hours, 4 hours, 4.5 hours, 5 hours, 5.5 hours, 6 hours, 7 hours, 8 hours, 9 hours, 10 hours, 11 hours, 12 hours, 18 hours, 24 hours, 48 hours, 72 hours, or a longer time period.

As used herein, a “suitably diluted” sample of a suitable fluid (e.g., plasma) includes, without limitation, a sample wherein the fluid is diluted by a factor of 4, a factor of 8, a factor of 16, a factor of 32, a factor of 64, a factor of 128, and a factor of 256.

As used herein, “treating” a labeled molecule (whether directly or indirectly) in a manner that facilitates its measurement includes, without limitation, (i) contacting the molecule with an antibody that specifically binds to the molecule (e.g., at the N-terminus, the C-terminus, a central domain, or any other domain); (ii) pre-treating the molecule (e.g., via acidification, alkalinization, heating, freezing, and/or sonication); and (ii) chemically treating the molecule (e.g., via phosphorylation, de-phosphorylation, oxidation, and/or oligomerization). Other agents, such as a dye or curcumin, can be used in place of antibodies for this purpose. Antibodies useful for treating a labeled molecule in a manner that facilitates its measurement include, without limitation, 3D6 (Johnson-Wood, et al. (1997), and Vanderstichele, et al. (2005)); 1E8 (Wiltfang, et al. (2001)); W02 (Vanderstichele, et al. (2005)); 82E1 (Horikoshia, et al. (2004)); 6E10 (Baghallab, et al. (2018) and Vanderstichele, et al. (2005)); 2G3 (Vanderstichele, et al. (2005)); and 21F12 (Johnson-Wood, et al. (1997), and Vanderstichele, et al. (2005)).

### Embodiments of the Invention

This invention provides certain methods for determining whether a subject has a condition correlative with a matrix effect. These conditions, such as amyloidopathy, can be diagnostic and/or prognostic for certain disorders, such as Alzheimer's disease.

Specifically, this invention provides a first method for determining whether a subject has a condition correlative with a matrix effect comprising (a) admixing (i) a suitably diluted sample of a suitable fluid from the subject and (ii) a suitable amount of a labeled molecule, wherein the labeled molecule is subject to a matrix effect with respect to the suitable fluid in a subject afflicted with the condition, and (b) after a suitable duration under suitable conditions, determining the amount of matrix-unaffected labeled molecule present in the resulting admixture, wherein the subject is afflicted with the condition if the amount of matrix-unaffected labeled molecule determined in step (b) correlates with a positive control for the condition, and wherein the subject is not afflicted with the condition if that amount correlates with a negative control for the condition.

In one embodiment of the first method, step (a) further comprises treating the labeled molecule in a manner that facilitates its measurement in step (b).

Preferably, treating the labeled molecule in a manner that facilitates its measurement in step (b) comprises contacting it with a suitable amount of an antibody that specifically binds to the labeled molecule.

In the first method, step (a) can be performed by admixing the suitable amount of labeled molecule either with a single diluted sample of the fluid, or with a plurality of serially diluted samples of the fluid.

This invention also provides a second method for determining whether a subject has a condition correlative with a matrix effect comprising (a) admixing (i) a suitably diluted sample of a suitable fluid from the subject and (ii) suitable amounts of a first labeled molecule and a second labeled molecule (and optionally three or more labeled molecules), wherein the labeled molecules are subject to a matrix effect with respect to the suitable fluid in a subject afflicted with the condition, and (b) after a suitable duration under suitable conditions, determining the quantitative relationship between matrix-unaffected first labeled molecule and matrix-unaffected second labeled molecule present in the resulting admixture, wherein the subject is afflicted with the condition if the relationship determined in step (b) correlates with a positive control for the condition, and wherein the subject is not afflicted with the condition if that relationship correlates with a negative control for the condition.

In one embodiment, the second method comprises (a) admixing (i) a suitably diluted sample of a suitable fluid from the subject and (ii) suitable amounts of three or more labeled molecules (e.g., labeled Aβ40 peptide, labeled Aβ42 peptide, and labeled Aβ10 peptide), wherein the labeled molecules are subject to a matrix effect with respect to the suitable fluid in a subject afflicted with the condition, and (b) after a suitable duration under suitable conditions, determining the quantitative relationship between each of the matrix-unaffected molecules present in the resulting admixture, wherein the subject is afflicted with the condition if the relationship determined in step (b) correlates with a positive control for the condition, and wherein the subject is not afflicted with the condition if that relationship correlates with a negative control for the condition.

In another embodiment of the second method, step (a) further comprises treating the labeled molecules in a manner that facilitates their measurement in step (b). Preferably, treating the labeled molecules in a manner that facilitates their measurement in step (b) comprises contacting each labeled molecule with a suitable amount of an antibody that specifically binds to it.

In the second method, step (a) can be performed by admixing the suitable amount of labeled molecules either with a single diluted sample of the fluid, or with a plurality of serially diluted samples of the fluid.

In the first and second methods, the subject is preferably a human.

In one embodiment of the first and second methods, the condition is a condition associated with a neurodegenerative disorder such as Alzheimer's disease, Parkinson's disease, mild cognitive impairment, non-Alzheimer's disease dementia, frontotemporal dementia, age-related macular degeneration (e.g., wet age-related macular degeneration), and dementia with Lewy bodies. In this embodiment, the subject can be either (i) symptomatic of Alzheimer's disease, Parkinson's disease, mild cognitive impairment, non-Alzheimer's disease dementia, frontotemporal dementia, age-related macular degeneration, or dementia with Lewy bodies, or (ii) asymptomatic of Alzheimer's disease, Parkinson's disease, mild cognitive impairment, non-Alzheimer's disease dementia, frontotemporal dementia, age-related macular degeneration, or dementia with Lewy bodies. In another embodiment of the first and second methods, the condition is a condition associated with Down's syndrome. In a further embodiment of the first and second methods, the condition is an amyloidopathy, a tauopathy, neuronal damage, a synucleinopathy, a cerebral proteinopathy (e.g., TDP proteinopathy), a progranulinopathy, and neuronal inflammation.

In the first and second methods, the suitable fluid is preferably whole blood, serum, plasma, cerebrospinal fluid, saliva, tear fluid, ocular fluid, vitreous humor, nasal fluid, and interstitial fluid.

In a preferred embodiment of the first method, the labeled molecule is a labeled Aβ peptide (e.g., labeled Aβ40 peptide, labeled Aβ42 peptide, labeled Aβ37 peptide, labeled Aβ38 peptide, labeled Aβ10 peptide, labeled Aβ16 peptide, or labeled Aβ43 peptide) or a fragment thereof, a labeled tau protein or a fragment thereof, a labeled phosphorylated tau protein or a fragment thereof, labeled progranulin or a fragment thereof, labeled TDP-43 or a fragment thereof, labeled GFAP or a fragment thereof, labeled neurogranin or a fragment thereof, labeled neurofilament or a fragment thereof, or a labeled synuclein (e.g., α-synuclein or β-synuclein) or a fragment thereof.

In a preferred embodiment of the second method, the first and second labeled molecules are selected from the group consisting of a labeled Aβ peptide (e.g., labeled Aβ40 peptide, labeled Aβ42 peptide, labeled Aβ37 peptide, labeled Aβ38 peptide, labeled Aβ10 peptide, labeled Aβ16 peptide, or labeled Aβ43 peptide) or a fragment thereof, a labeled tau protein or a fragment thereof, a labeled phosphorylated tau protein or a fragment thereof, labeled progranulin or a fragment thereof, labeled TDP-43 or a fragment thereof, labeled GFAP or a fragment thereof, labeled neurogranin or a fragment thereof, labeled neurofilament or a fragment thereof, and a labeled synuclein (e.g., α-synuclein or β-synuclein) or a fragment thereof.

In the first and second methods, determining the amount of matrix-unaffected labeled molecule can be accomplished in absolute terms (e.g., in pg/ml or total pg) or comparative terms (e.g., in ppm or fold difference relative to another molecule).

In this invention, a method is envisioned for measuring the progression in a subject of a condition correlative with a matrix effect comprising (a) performing the first method at a plurality of time points (e.g., one month apart, two months apart, or three months apart), and (b) comparing with each other the amounts of matrix-unaffected labeled molecule determined by the first method at each of the time points. In one embodiment of this method, the subject is undergoing treatment for the condition, and the method is used to monitor the success of the treatment. In this invention, another method is envisioned for measuring the progression in a subject of a condition correlative with a matrix effect comprising (a) performing the second method at a plurality of time points (e.g., one month apart, two months apart, or three months apart), and (b) comparing with each other the amounts of matrix-unaffected labeled molecule determined by the second method at each of the time points. In one embodiment of this method, the subject is undergoing treatment for the condition, and the method is used to monitor the success of the treatment. The various embodiments of these condition progression-measuring methods are envisioned, mutatis mutandis, as they are for the first and second methods of this invention.

This invention further provides a first kit for use in determining whether a subject (preferably a human subject) has a condition correlative with a matrix effect comprising, in separate compartments, (a) a labeled molecule, wherein the labeled molecule is subject to a matrix effect with respect to a suitable fluid in a subject afflicted with the condition, and (b) an agent useful for treating the labeled molecule to facilitate its measurement in matrix-unaffected form. In a preferred embodiment of the first kit, the kit further comprises, in a separate compartment, an agent useful for measuring the labeled molecule in its matrix-unaffected form. In another preferred embodiment of the first kit, the labeled molecule is selected from the group consisting of a labeled Aβ peptide (e.g., labeled Aβ40 peptide, labeled Aβ42 peptide, labeled Aβ37 peptide, labeled Aβ38 peptide, labeled Aβ10 peptide, labeled Aβ16 peptide, or labeled Aβ43 peptide) or a fragment thereof, a labeled tau protein or a fragment thereof, a labeled phosphorylated tau protein or a fragment thereof, labeled progranulin or a fragment thereof, labeled TDP-43 or a fragment thereof, labeled GFAP or a fragment thereof, labeled neurogranin or a fragment thereof, labeled neurofilament or a fragment thereof, and a labeled synuclein (e.g., α-synuclein) or a fragment thereof.

This invention still further provides a second kit for use in determining whether a subject (preferably a human subject) has a condition correlative with a matrix effect comprising, in separate compartments, (a) a first labeled molecule and a second labeled molecule, wherein each labeled molecule is subject to a matrix effect with respect to a suitable fluid in a subject afflicted with the condition, and (b) an agent useful for treating each of the first and second labeled molecules to facilitate its measurement in matrix-unaffected form. In a preferred embodiment of the second kit, the kit further comprises, in one or more separate compartments, an agent useful for measuring each of the first and second labeled molecules in its matrix-unaffected form. In another preferred embodiment of the second kit, the first and second labeled molecules are selected from the group consisting of a labeled Aβ peptide (e.g., labeled Aβ40 peptide, labeled Aβ42 peptide, labeled Aβ37 peptide, labeled Aβ38 peptide, labeled Aβ10 peptide, labeled Aβ16 peptide, or labeled Aβ43 peptide) or a fragment thereof, a labeled tau protein or a fragment thereof, a labeled phosphorylated tau protein or a fragment thereof, labeled progranulin or a fragment thereof, labeled TDP-43 or a fragment thereof, labeled GFAP or a fragment thereof, labeled neurogranin or a fragment thereof, labeled neurofilament or a fragment thereof, and a labeled synuclein (e.g., α-synuclein) or a fragment thereof.

In one embodiment, the second kit comprises, in separate compartments, (a) three or more labeled molecules (e.g., labeled Aβ40 peptide, labeled Aβ42 peptide, and labeled Aβ10 peptide), wherein each labeled molecule is subject to a matrix effect with respect to a suitable fluid in a subject afflicted with the condition, and (b) one or more agents useful for treating the labeled molecules to facilitate their measurement in matrix-unaffected form.

This invention provides a first method for determining whether a human subject (either symptomatic or asymptomatic) has a condition correlative with Alzheimer's disease (i.e., wherein the subject may or may not actually be afflicted yet with Alzheimer's disease) comprising (a) admixing (i) a suitably diluted sample of a suitable fluid from the subject and (ii) a suitable amount of a labeled Aβ peptide, and (b) after a suitable duration under suitable conditions, determining the amount of matrix-unaffected labeled Aβ peptide present in the resulting admixture, wherein the subject is afflicted with the condition if the amount of matrix-unaffected labeled Aβ peptide determined in step (b) correlates with a positive control for the condition, and wherein the subject is not afflicted with the condition if that amount correlates with a negative control for the condition.

In one embodiment, the first method comprises (a) admixing (i) a first suitably diluted sample of a suitable fluid from the subject and (ii) a suitable amount of a labeled Aβ peptide, and separately admixing (i) a second suitably diluted sample of a suitable fluid from the subject and (ii) a suitable amount of a labeled Aβ peptide, (b) after a suitable duration under suitable conditions, determining the quantitative relationship between matrix-unaffected labeled Aβ peptide at the first dilution and matrix-unaffected labeled Aβ peptide at the second dilution present in the resulting admixtures, wherein the subject is afflicted with the condition if the relationship determined in step (b) correlates with a positive control for the condition, and wherein the subject is not afflicted with the condition if that relationship correlates with a negative control for the condition. For example, in one embodiment, the relationship determined is the ratio of matrix-unaffected labeled Aβ peptide measured for a 1:8 diluted plasma sample (the first suitably diluted sample) to matrix-unaffected labeled Aβ peptide measured for a 1:128 diluted plasma sample (the second suitably diluted sample).

In another embodiment of the first method, step (a) further comprises treating the labeled Aβ peptide in a manner that facilitates its measurement in step (b).

Preferably, treating the labeled Aβ peptide in a manner that facilitates its measurement in step (b) comprises contacting it with a suitable amount of an agent (such as an antibody) that binds (preferably specifically) to the labeled Aβ peptide. Such agents include, without limitation, antibody 3D6 (Johnson-Wood, et al. (1997), and Vanderstichele, et al. (2005)); antibody 1E8 (Wiltfang, et al. (2001)); antibody W02 (Vanderstichele, et al. (2005)); antibody 82E1 (Horikoshia, et al. (2004)); antibody 6E10 (Baghallab, et al. (2018) and Vanderstichele, et al. (2005)); antibody 2G3 (Vanderstichele, et al. (2005)); and antibody 21F12 (Johnson-Wood, et al. (1997), and Vanderstichele, et al. (2005)).

In the first method, step (a) can be performed by admixing the suitable amount of labeled Aβ peptide either with a single diluted sample of the fluid, or with a plurality of serially diluted samples of the fluid.

In a preferred embodiment of the first method, the labeled Aβ peptide is labeled Aβ40 peptide or a fragment thereof, labeled Aβ42 peptide or a fragment thereof, labeled Aβ37 peptide or a fragment thereof, labeled Aβ38 peptide or a fragment thereof, labeled Aβ10 peptide or a fragment thereof, labeled Aβ16 peptide or a fragment thereof, or labeled Aβ43 peptide or a fragment thereof. In a further preferred embodiment of the first method, the labeled Aβ peptide is biotinylated Aβ40 peptide, biotinylated Aβ42 peptide, or a fragment thereof.

This invention also provides a second method for determining whether a human subject (either symptomatic or asymptomatic) has a condition correlative with Alzheimer's disease (i.e., wherein the subject may or may not actually be afflicted yet with Alzheimer's disease) comprising (a) admixing (i) a suitably diluted sample of a suitable fluid from the subject and (ii) suitable amounts of a first labeled Aβ peptide and a second labeled Aβ peptide, and (b) after a suitable duration under suitable conditions, determining the quantitative relationship between matrix-unaffected first labeled Aβ peptide and matrix-unaffected second labeled Aβ peptide present in the resulting admixture, wherein the subject is afflicted with the condition if the relationship determined in step (b) correlates with a positive control for the condition, and wherein the subject is not afflicted with the condition if that relationship correlates with a negative control for the condition.

In one embodiment of the second method, step (a) further comprises treating the labeled Aβ peptides in a manner that facilitates their measurement in step (b). Preferably, treating the labeled Aβ peptides in a manner that facilitates their measurement in step (b) comprises contacting it with a suitable amount of an agent (such as an antibody) that binds (preferably specifically) to the labeled Aβ peptide. Such agents include, without limitation, antibody 3D6 (Johnson-Wood, et al. (1997), and Vanderstichele, et al. (2005)); antibody 1E8 (Wiltfang, et al. (2001)); antibody W02 (Vanderstichele, et al. (2005)); antibody 82E1 (Horikoshia, et al. (2004)); antibody 6E10 (Baghallab, et al. (2018) and Vanderstichele, et al. (2005)); antibody 2G3 (Vanderstichele, et al. (2005)); and antibody 21F12 (Johnson-Wood, et al. (1997), and Vanderstichele, et al. (2005)).

In the second method, step (a) can be performed by admixing the suitable amounts of labeled Aβ peptides either with a single diluted sample of the fluid, or with a plurality of serially diluted samples of the fluid.

In a preferred embodiment of the second method, each of the labeled Aβ peptides is labeled Aβ40 peptide or a fragment thereof, labeled Aβ42 peptide or a fragment thereof, labeled Aβ37 peptide or a fragment thereof, labeled Aβ38 peptide or a fragment thereof, labeled Aβ10 peptide or a fragment thereof, labeled Aβ316 peptide or a fragment thereof, or labeled Aβ43 peptide or a fragment thereof. In a further preferred embodiment of the second method, each of the labeled Aβ peptides is biotinylated Aβ40 peptide, biotinylated Aβ42 peptide, or a fragment thereof.

In the first and second methods, the condition correlative with Alzheimer's disease can be any such condition having that correlation while also being correlative with a matrix effect. Preferably, the condition is an amyloidopathy, a tauopathy, neuronal damage, a synucleinopathy, a cerebral proteinopathy (e.g., TDP-43), a progranulinopathy, and neuronal inflammation.

In the first and second methods, the suitable fluid is preferably whole blood, serum, plasma, cerebrospinal fluid, saliva, tear fluid, ocular fluid, vitreous humor, nasal fluid, or interstitial fluid.

In the first and second methods, determining the amount of matrix-unaffected labeled molecule can be accomplished in absolute terms (e.g., in pg/ml or total pg) or comparative terms (e.g., in ppm or fold difference relative to another molecule).

In this invention, a method is envisioned for measuring the progression in a subject of a condition correlative with Alzheimer's disease comprising (a) performing the first method at a plurality of time points (e.g., one month apart, two months apart, or three months apart), and (b) comparing with each other the amounts of matrix-unaffected labeled Aβ peptide determined by the first method at each of the time points. In one embodiment of this method, the subject is undergoing treatment for the condition, and the method is used to monitor the success of the treatment. In this invention, another method is envisioned for measuring the progression in a subject of a condition correlative with Alzheimer's disease comprising (a) performing the second method at a plurality of time points (e.g., one month apart, two months apart, or three months apart), and (b) comparing with each other the amounts of matrix-unaffected labeled Aβ peptide determined by the second method at each of the time points. In one embodiment of this method, the subject is undergoing treatment for the condition, and the method is used to monitor the success of the treatment. The various embodiments of these condition progression-measuring methods are envisioned, mutatis mutandis, as they are for the first and second methods of this invention.

This invention further provides a first kit for use in determining whether a human subject has a condition correlative with Alzheimer's disease comprising, in separate compartments, (a) a labeled Aβ peptide, and (b) an agent useful for treating the labeled Aβ peptide to facilitate the labeled Aβ peptide's measurement in its matrix-unaffected form. In a preferred embodiment of the first kit, the kit further comprises, in a separate compartment, an agent useful for measuring the labeled Aβ peptide in its matrix-unaffected form. In another preferred embodiment of the first kit, the labeled Aβ peptide is labeled Aβ40 peptide or a fragment thereof, labeled Aβ42 peptide or a fragment thereof, labeled Aβ37 peptide or a fragment thereof, labeled Aβ38 peptide or a fragment thereof, labeled Aβ10 peptide or a fragment thereof, labeled Aβ16 peptide or a fragment thereof, or labeled Aβ43 peptide or a fragment thereof.

In a further preferred embodiment of the first kit, the labeled Aβ peptide is biotinylated Aβ40 peptide or biotinylated Aβ42 peptide, or a fragment thereof.

This invention still further provides a second kit for use in determining whether a human subject has a condition correlative with Alzheimer's disease comprising, in separate compartments, (a) a first labeled Aβ peptide and a second labeled Aβ peptide, and (b) an agent useful for treating each of the first and second labeled Aβ peptides to facilitate the labeled Aβ peptide's measurement in its matrix-unaffected form. In a preferred embodiment of the second kit, the kit further comprises, in one or more separate compartments, an agent useful for measuring each of the first and second labeled Aβ peptides in its matrix-unaffected form. In another preferred embodiment of the second kit, each of the labeled Aβ peptides is labeled Aβ40 peptide or a fragment thereof, labeled Aβ42 peptide or a fragment thereof, labeled Aβ37 peptide or a fragment thereof, labeled Aβ38 peptide or a fragment thereof, labeled Aβ10 peptide or a fragment thereof, labeled Aβ16 peptide or a fragment thereof, or labeled Aβ43 peptide or a fragment thereof. In a further preferred embodiment of the second kit, the first and second labeled Aβ peptides are biotinylated Aβ40 peptide and biotinylated Aβ42 peptide, or a fragment thereof.

This invention provides a first method for determining whether a human subject (either symptomatic or asymptomatic) has a condition correlative with Parkinson's disease (i.e., wherein the subject may or may not actually be afflicted yet with Parkinson's disease) comprising (a) admixing (i) a suitably diluted sample of a suitable fluid from the subject and (ii) a suitable amount of a labeled α-synuclein, and (b) after a suitable duration under suitable conditions, determining the amount of matrix-unaffected labeled α-synuclein present in the resulting admixture, wherein the subject is afflicted with the condition if the amount of matrix-unaffected labeled α-synuclein determined in step (b) correlates with a positive control for the condition, and wherein the subject is not afflicted with the condition if that amount correlates with a negative control for the condition.

In one embodiment, the first method comprises (a) admixing (i) a first suitably diluted sample of a suitable fluid from the subject and (ii) a suitable amount of a labeled α-synuclein, and separately admixing (i) a second suitably diluted sample of a suitable fluid from the subject and (ii) a suitable amount of a labeled α-synuclein, (b) after a suitable duration under suitable conditions, determining the quantitative relationship between matrix-unaffected labeled α-synuclein at the first dilution and matrix-unaffected labeled α-synuclein at the second dilution present in the resulting admixtures, wherein the subject is afflicted with the condition if the relationship determined in step (b) correlates with a positive control for the condition, and wherein the subject is not afflicted with the condition if that relationship correlates with a negative control for the condition. For example, in one embodiment, the relationship determined is the ratio of matrix-unaffected labeled α-synuclein measured for a 1:8 diluted plasma sample (the first suitably diluted sample) to matrix-unaffected labeled α-synuclein measured for a 1:128 diluted plasma sample (the second suitably diluted sample).

In another embodiment of the first method, step (a) further comprises treating the labeled α-synuclein in a manner that facilitates its measurement in step (b).

Preferably, treating the labeled α-synuclein in a manner that facilitates its measurement in step (b) comprises contacting it with a suitable amount of an antibody that specifically binds to the labeled α-synuclein.

In the first method, step (a) can be performed by admixing the suitable amount of labeled α-synuclein either with a single diluted sample of the fluid, or with a plurality of serially diluted samples of the fluid.

This invention also provides a second method for determining whether a human subject (either symptomatic or asymptomatic) has a condition correlative with Parkinson's disease (i.e., wherein the subject may or may not actually be afflicted yet with Parkinson's disease) comprising (a) admixing (i) a suitably diluted sample of a suitable fluid from the subject and (ii) suitable amounts of a first labeled α-synuclein and a second labeled α-synuclein, and (b) after a suitable duration under suitable conditions, determining the quantitative relationship between matrix-unaffected first labeled α-synuclein and matrix-unaffected second labeled α-synuclein present in the resulting admixture, wherein the subject is afflicted with the condition if the relationship determined in step (b) correlates with a positive control for the condition, and wherein the subject is not afflicted with the condition if that relationship correlates with a negative control for the condition.

In one embodiment of the second method, step (a) further comprises treating the labeled α-synucleins in a manner that facilitates their measurement in step (b). Preferably, treating the labeled α-synucleins in a manner that facilitates their measurement in step (b) comprises contacting them with a suitable amount of an antibody that specifically binds to the labeled α-synucleins.

In the second method, step (a) can be performed by admixing the suitable amounts of labeled α-synucleins either with a single diluted sample of the fluid, or with a plurality of serially diluted samples of the fluid.

In the first and second methods, the suitable fluid is preferably whole blood, serum, plasma, cerebrospinal fluid, saliva, tear fluid, nasal fluid, ocular fluid, vitreous humor, or interstitial fluid.

In the first and second methods, determining the amount of matrix-unaffected labeled molecule can be accomplished in absolute terms (e.g., in pg/ml or total pg) or comparative terms (e.g., in ppm or fold difference relative to another molecule).

In this invention, a method is envisioned for measuring the progression in a subject of a condition correlative with Parkinson's disease comprising (a) performing the first method at a plurality of time points (e.g., one month apart, two months apart, or three months apart), and (b) comparing with each other the amounts of matrix-unaffected labeled α-synuclein determined by the first method at each of the time points. In one embodiment of this method, the subject is undergoing treatment for the condition, and the method is used to monitor the success of the treatment. In this invention, another method is envisioned for measuring the progression in a subject of a condition correlative with Parkinson's disease comprising (a) performing the second method at a plurality of time points (e.g., one month apart, two months apart, or three months apart), and (b) comparing with each other the amounts of matrix-unaffected labeled α-synuclein determined by the second method at each of the time points. In one embodiment of this method, the subject is undergoing treatment for the condition, and the method is used to monitor the success of the treatment. The various embodiments of these condition progression-measuring methods are envisioned, mutatis mutandis, as they are for the first and second methods of this invention.

This invention further provides a first kit for use in determining whether a human subject has a condition correlative with Parkinson's disease comprising, in separate compartments, (a) a labeled α-synuclein, and (b) an agent useful for treating the labeled α-synuclein to facilitate the labeled α-synuclein's measurement in its matrix-unaffected form. In a preferred embodiment of the first kit, the kit further comprises, in a separate compartment, an agent useful for measuring the labeled α-synuclein in its matrix-unaffected form. In a further preferred embodiment of the first kit, the labeled α-synuclein is biotinylated α-synuclein or a fragment thereof.

This invention still further provides a second kit for use in determining whether a human subject has a condition correlative with Parkinson's disease comprising, in separate compartments, (a) a first labeled α-synuclein and a second labeled α-synuclein, and (b) an agent useful for treating each of the first and second labeled α-synucleins to facilitate the labeled α-synucleins' measurement in their matrix-unaffected form. In a preferred embodiment of the second kit, the kit further comprises, in one or more separate compartments, an agent useful for measuring each of the first and second labeled α-synucleins in their matrix-unaffected form. In a further preferred embodiment of the second kit, the first and second labeled α-synucleins are biotinylated α-synucleins, or fragments thereof.

In the above first and second methods and first and second kits for use in connection with Parkinson's disease, α-synuclein may be substituted with one or more phosphoproteins, neurogranin, or one or more Rab proteins.

Finally, this invention provides certain compositions of matter. A first composition comprises an admixture of a labeled Aβ peptide (e.g., a biotinylated Aβ peptide) and a diluted fluid sample from a human subject. In one embodiment, the composition comprises (i) an admixture of biotinylated Aβ37 peptide (or a fragment thereof) and a diluted human plasma sample; (ii) an admixture of biotinylated Aβ38 peptide (or a fragment thereof) and a diluted human plasma sample; (iii) an admixture of biotinylated Aβ40 peptide (or a fragment thereof) and a diluted human plasma sample; (iv) an admixture of biotinylated Aβ42 peptide (or a fragment thereof) and a diluted human plasma sample; (v) an admixture of biotinylated Aβ43 peptide (or a fragment thereof) and a diluted human plasma sample; (vi) an admixture of biotinylated Aβ10 peptide (or a fragment thereof) and a diluted human plasma sample; or (vii) an admixture of biotinylated Aβ16 peptide (or a fragment thereof) and a diluted human plasma sample. A second composition comprises an admixture of a labeled Aβ peptide (or a fragment thereof), an antibody that specifically binds to the labeled Aβ peptide (or fragment thereof), and a diluted fluid sample from a human subject. In one embodiment, the composition comprises (i) an admixture of biotinylated Aβ37 peptide (or a fragment thereof), an antibody that specifically binds to the labeled Aβ peptide (or fragment thereof), and a diluted human plasma sample; (ii) an admixture of biotinylated Aβ38 peptide (or a fragment thereof), an antibody that specifically binds to the labeled Aβ peptide (or fragment thereof), and a diluted human plasma sample; (iii) an admixture of biotinylated Aβ40 peptide (or a fragment thereof), an antibody that specifically binds to the labeled Aβ peptide (or fragment thereof), and a diluted human plasma sample; (iv) an admixture of biotinylated Aβ42 peptide (or a fragment thereof), an antibody that specifically binds to the labeled Aβ peptide (or fragment thereof), and a diluted human plasma sample; (v) an admixture of biotinylated Aβ43 peptide (or a fragment thereof), an antibody that specifically binds to the biotinylated Aβ peptide (or fragment thereof), and a diluted human plasma sample; (vi) an admixture of biotinylated Aβ10 peptide (or a fragment thereof), an antibody that specifically binds to the biotinylated Aβ peptide (or fragment thereof), and a diluted human plasma sample; or (vii) an admixture of biotinylated Aβ16 peptide (or a fragment thereof), an antibody that specifically binds to the biotinylated Aβ peptide (or fragment thereof), and a diluted human plasma sample.

A third composition comprises an admixture of a labeled tau protein (e.g., a biotinylated tau protein) (or a fragment thereof) and a diluted fluid sample from a human subject. In one embodiment, the composition comprises an admixture of biotinylated tau protein (or a fragment thereof) and a diluted human plasma sample. A fourth composition comprises an admixture of a labeled tau protein (or a fragment thereof), an antibody that specifically binds to the labeled tau protein (or fragment thereof), and a diluted fluid sample from a human subject. In one embodiment, the composition comprises an admixture of biotinylated tau protein (or a fragment thereof), an antibody that specifically binds to the biotinylated tau protein (or fragment thereof), and a diluted human plasma sample.

A fifth composition comprises an admixture of a labeled phosphorylated tau protein (e.g., a biotinylated phosphorylated tau protein) (or a fragment thereof) and a diluted fluid sample from a human subject. In one embodiment, the composition comprises an admixture of biotinylated phosphorylated tau protein (or a fragment thereof) and a diluted human plasma sample. A sixth composition comprises an admixture of a labeled phosphorylated tau protein (or a fragment thereof), an antibody that specifically binds to the labeled phosphorylated tau protein (or fragment thereof), and a diluted fluid sample from a human subject. In one embodiment, the composition comprises an admixture of biotinylated phosphorylated tau protein (or a fragment thereof), an antibody that specifically binds to the biotinylated phosphorylated tau protein (or fragment thereof), and a diluted human plasma sample.

A seventh composition comprises an admixture of a diluted fluid sample from a human subject and a labeled synuclein (e.g., a biotinylated α-synuclein) (or a fragment thereof). In one embodiment, the composition comprises an admixture of biotinylated α-synuclein (or a fragment thereof) and a diluted human plasma sample. An eighth composition comprises an admixture of a labeled synuclein (or a fragment thereof), an antibody that specifically binds to the labeled synuclein (or fragment thereof), and a diluted fluid sample from a human subject. In one embodiment, the composition comprises an admixture of biotinylated α-synuclein (or a fragment thereof), an antibody that specifically binds to the biotinylated α-synuclein (or fragment thereof), and a diluted human plasma sample.

A ninth composition comprises an admixture of a diluted fluid sample from a human subject and a labeled progranulin (e.g., a biotinylated progranulin) (or a fragment thereof). A tenth composition comprises an admixture of a diluted fluid sample from a human subject and a labeled TDP-43 (e.g., a biotinylated TDP-43) (or a fragment thereof). An eleventh composition comprises an admixture of a diluted fluid sample from a human subject and a labeled GFAP (e.g., a biotinylated GFAP) (or a fragment thereof). A twelfth composition comprises an admixture of a diluted fluid sample from a human subject and a labeled neurogranin (e.g., a biotinylated neurogranin) (or a fragment thereof). A thirteenth composition comprises an admixture of a diluted fluid sample from a human subject and a labeled neurofilament (e.g., a biotinylated neurofilament) (or a fragment thereof).

The present methods and kits are envisioned for tau protein-based methods and kits, phosphorylated tau protein-based methods and kits, and synuclein-based methods and kits, mutatis mutandis, as they are for Aβ peptide-based methods and kits in this invention.

The present methods and kits are envisioned for unlabeled molecules (e.g., α-synuclein), mutatis mutandis, as they are for labeled molecules in this invention. The present methods and kits are also envisioned for determining whether a human subject (either symptomatic or asymptomatic) has a condition correlative with cognitive decline, mutatis mutandis, as they are for determining whether a human subject (either symptomatic or asymptomatic) has a condition correlative with Alzheimer's disease in this invention.

This invention will be better understood by reference to the examples which follow, but those skilled in the art will readily appreciate that the specific examples detailed are only illustrative of the invention as described more fully in the claims which follow thereafter.

### EXAMPLES

### Example 1—the Technology

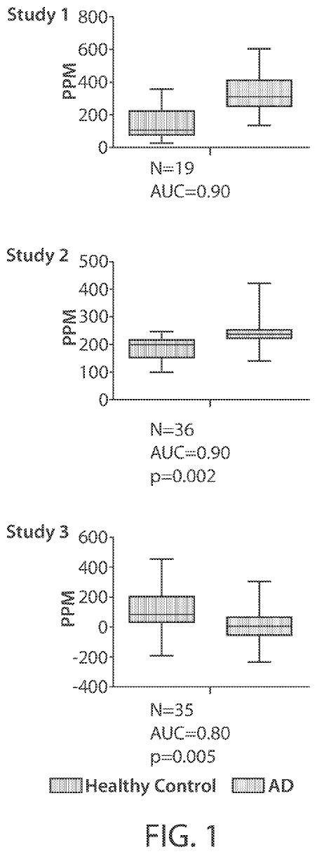

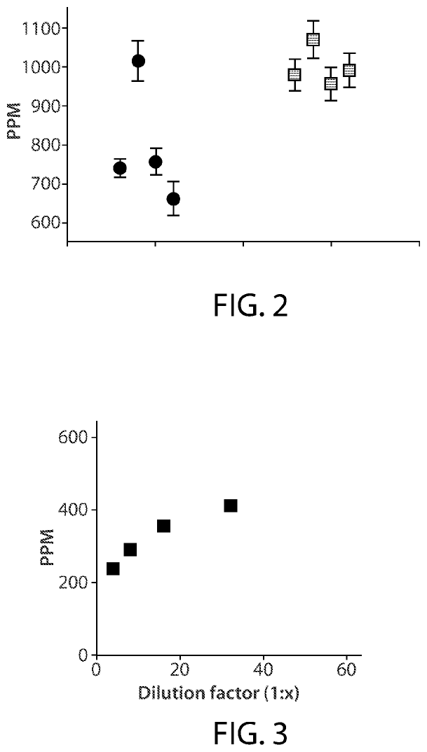

All data in FIG. 1 were generated on the MagArray technology (Milpitas, California). The assay was run on a MagArray MR-813 instrument system (i.e., a Hanno incubation unit, and a Reader unit). MagArray Giant Magneto-resistive (GMR) sensor technology and associated instruments enable ultra-sensitive detection of low abundance biomolecules in a multiplexed format that requires no optics or microfluidics, while providing a simultaneous real-time readout (J. R. Lee, et al. (2017) and R. S. Gaster, et al. (2009)).

The assay formats used in the present study are configured with one or more monoclonal antibodies (mAbs) spotted on sensors on printed circuit boards (PCB). Each PCB contains 8 chips. Each chip contains 80-sensors that can be spotted with the same or different antibodies. Several sensors were spotted with the same antibody in order to obtain a more robust and accurate signal per analyte or target antigen. Bound analytes (biotinylated peptides) were detected using custom magnetic nanotechnology (anti-biotin antibodies coupled to magnetic nanoparticles (MNP) obtained from Miltenyi Biotec (California, USA). All PCBs were surface-treated and prepared by MagArray using a proprietary process. After PCB surface treatments, antibodies were spotted, and these pre-functionalized PCBs were used either freshly prepared, frozen fresh, or baked and frozen. If stored frozen, PCBs were brought back to room temperature (RT) before being used in the assay. Some sensors were spotted with bovine serum albumin as a reference protein. Reference protein sensor signals were used to normalize for chip-specific variability in sensor rows and columns, while the empty sensors allowed for assessment of non-specific signal in a clinical sample.

### Example 2—Assay 1

The assay was run on a MagArray MR-813 instrument system (i.e., a Hanno incubation unit, and a Reader unit). Each plasma sample was serially diluted with a phosphate buffer saline (PBS) solution containing albumin as the stabilizing buffer solution. Each component was prepared in the same PBS buffer. In brief, the process includes a pre-incubation step on an orbital shaker of pre-diluted EDTA-plasma samples (Dilution factor: ⅛> 1/1024) with specific concentrations of N-terminally biotinylated Aβ1-40 or Aβ1-42 peptides (rPeptide, Watkinsville, Georgia, US), followed by incubation for two hours at room temperature on a Hanno unit where plasma samples are exposed to capture antibodies on the chips. PCBs are then taken to the Reader unit and immersed in the MNP reagent in the presence of a magnetic field for 20-30 minutes, during which signals from each GMR sensor are obtained.

Antibodies

Monoclonal antibodies were obtained from different sources. The characteristics of antibodies 21F12, 2G3, and 3D6 were described previously (Johnson-Wood, et al. (1997) and Bard, et al. (2003)). In addition, epitope-mapping experiments were documented in Vanderstichele, et al. (2005).

Samples

EDTA-plasma samples were obtained from PrecisionMed (Solana Beach, California, US), GoldonWest Biosolutions (Temecula, California, USA) or from University Centers. Detailed information on gender, age, mini mental status examinations (MMSE), and CSF biomarker profiles were available at the time of ordering the samples. Samples were shipped on dry ice and stored immediately at <−20° C. upon arrival in the lab. During preparation of samples or aliquotation, care was taken to always use polypropylene recipients with a low protein adsorption capacity.

Three different sample sets have been used (Study 1, Study 2, Study 3). Each sample set was divided into EDTA-plasma obtained from healthy controls or subjects with a diagnosis of Alzheimer's disease. The number of subjects differ as a function of the study protocol, as well as with respect to the study design or the outcome of the assay (See Table 1).

Each sample was serially diluted with a PBS solution containing albumin as the stabilizing buffer solution.

TABLE 1

| Study | | 1 | 2 | 3 |
|---|---|---|---|---|
| Samples | Source | Precision- | Precision- | Precision- |
| | | Med | Med | Med |
| | Number | 19 | 36 | 35 |
| | Type | EDTA-Plasma | | |
| Outcome | Isoform | Aβ40 | Aβ42 | Aβ42 |
| | Parameter | PPM | PPM | PPM |
| | Plasma Dilution | 32 | 256 | Algorithm |
| | factor (DF) | | | DF64-DF128 |
| Result | Area Under the | 0.90 | 0.9 | 0.8 |
| | Curve | | | |
| | p-Value | Not available | 0.002 | 0.005 |
| Assay | Capture mAb | 2G3 | 21F12 | 21F12 |
| format | Detection | Anti-biotin- | Anti-biotin- | Anti-biotin- |
| | conjugate | MNP | MNP | MNP |
| | Reading time | 23 | 23 | 23 |
| | (min) | | | |

### Example 3—Assay 2

The assay protocol was run on a MagArray MR-813 instrument system. Each sample was serially diluted with a PBS solution containing albumin as the stabilizing buffer solution. Each component was prepared in the same PBS buffer. In brief, the process includes a pre-incubation step on an orbital shaker of pre-diluted EDTA-plasma samples (Dilution factor: ⅛- 1/1024) with specific concentrations of Aβ1-10-bio (AnaSpec, Fremont, California, USA), followed by incubation for two hours at room temperature on a Hanno unit where plasma samples are exposed to capture antibodies on the chips. PCBs are then taken to the Reader unit and immersed in the MNP reagent in the presence of a magnetic field for 20-30 minutes, during which signals from each GMR sensor are obtained.

Antibodies

The characteristics of antibodies 21F12, 2G3, and 3D6 were described previously (Johnson-Wood, et al. (1997) and Bard, et al. (2003)). In addition, epitope mapping experiments were documented in Vanderstichele, et al. 2005.

Samples

EDTA-plasma samples were obtained from PrecisionMed (Solana Beach, California, US), GoldonWest Biosolutions (Temecula, California, USA), or from University Centers. Detailed information on gender, age, MMSE, and CSF biomarker profiles were available at the time of ordering the materials. Materials were shipped on dry ice and stored immediately at <−20° C. upon arrival in the lab. During preparation of samples or aliquotation, care was taken to always use polypropylene recipients with low protein adsorption capacity. Four EDTA-plasma samples obtained from healthy controls and four subjects with diagnosis of Alzheimer's disease were used in the study. Each sample was serially diluted with a PBS solution containing albumin as the stabilizing agent. Each sample was tested individually on four different boards coated with 3D6, together with other mAbs that are used as negative controls, internal controls, and run validation controls.

### Example 4—Assay 3

Boards were prepared using a mixture of tau- or phospho-tau-specific capture antibodies. Monoclonal antibodies were obtained from Thermo Fisher Scientific (Waltham, MA, USA). In this example, AT270, a mAb specific for tau phosphorylated at position 181, was used as capture antibody, while biotinylated HT7 (Epitope: 159-163 of the tau protein) was used as detection antibody. The characteristics of these mAbs were published previously (E. Vanmechelen, et al. (2000) and H. Vanderstichele, et al. (2006)). The EDTA-plasma sample was obtained from GoldonWest Biosolutions (Temecula, California, USA). Samples were shipped on dry ice and stored immediately at <−20° C. upon arrival in the lab. During preparation of the different dilutions of the plasma, care was taken to always use polypropylene recipients with a low protein adsorption capacity.

The assay was run on a MagArray MR-813 instrument system. All components (peptides, diluted plasma, and biotinylated antibodies) in the assay were prepared using the same PBS buffer. Plasma sample was serially diluted (from 1:2 to 1:32) with a PBS containing albumin as a stabilizer protein. 240 μL of the pre-diluted plasma was mixed with 10 μL of buffer containing 2,500 pg/mL of a synthetic peptide containing the epitope of HT7 and phosphorylated at position 181 (Obtained from Proteogenix, Schiltigheim, France). The added peptide was non-biotinylated. Thereafter, an equal volume of buffer with biotinylated HT7 (200 ng/mL) was added. The mixture was pre-incubated at room temperature for 1 h on a Hanno unit followed by an incubation of 4 hours with the capture antibodies on the PCB. At the end of the incubation period, the PCB was immersed in the MNP reagent in the presence of a magnetic field for 20-30 minutes, during which signals from each GMR sensor were obtained.

### Example 5—Amino Acid Sequences of Certain Human A13 Peptides

This Example sets forth the amino acid sequences of certain human Aβ peptides.

The amino acid sequence of Aβ1-37 is as follows: 1-DAEFRHDSGYEVHHQKL-VFFAEDVGSNKGAIIGLMVG-37. The amino acid sequence of Aβ1-38 is as follows: 1-DAEFRHDSGYEVHHQKLVFFAEDVGSNKGAIIGLMVGG-38. The amino acid sequence of Aβ1-40 is as follows: 1-DAEFRHDSGYEVHHQKL-VFFAEDVGSNKGAIIGLMVGGW-40. The amino acid sequence of Aβ1-42 is as follows: 1-DAEFRHDSGYEVHHQKLVFFAEDVGSNKGAIIGLMVGGVVIA-42. The amino acid sequence of Aβ43 is as follows: 1-DAEFRHDSGYEVHHQKLV-FFAEDVGSNKGAIIGLMVGGVVIAT-43. The amino acid sequence of Aβ-3-40 is as follows: (-3)-VKMDAEFRHDSGYEVHHQKLVFFAEDVGSNKGAIIGLMV-GGW-40.

### REFERENCES

I. Baghallab, et al., Epitomic Characterization of the Specificity of the Anti-Amyloid A Monoclonal Antibodies 6E10 and 4G8, Journal of Alzheimer's Disease 66 (2018) 1235-1244 DOI 10.3233/JAD-180582.
F. Bard, et al., Epitope and isotype specificities of antibodies to beta-amyloid peptide for protection against Alzheimer's disease-like neuropathology. Proc Nat/Acad Sci USA 2003; 100:2023-2028.
S. Barrio, Methods and reagents for improved detection of amyloid beta peptides, U.S. Publication No. 2012/0264642 (Oct. 18, 2012).
M. Bjerke, et al., Confounding Factors Influencing Amyloid Beta Concentration in Cerebrospinal Fluid, Int'l J. of Alzheimer's Disease (2010), https://doi.org/10.4061/2010/986310.
C. Carroll, et al., Heart-type fatty acid binding protein as an early marker for myocardial infarction: systematic review and meta-analysis. J. Emerg Med 2013 (30):280-286.
V. C. Cullen, et al., Development and Advanced Validation of an Optimized Method for the Quantitation of Aβ₄₂ in Human Cerebrospinal Fluid, The AAPS Journal (2012), 14, 510-518.
R. S. Gaster, et al., Matrix-insensitive protein assays push the limits of biosensors in medicine. Nat Med. 2009 Nov.; 15(11):1327-32. doi: 10.1038/nm.2032).
N. P. Groome, et al., Region-specific immunoassays for human myelin basic protein, 1986, J. Neuroimmunol 12: 253-264.
M. Gu and J. Viles, Methionine oxidation reduces lag-times for amyloid-β(1-40) fiber formation but generates highly fragmented fibers, Biochimica et Biophysica Acta (BBA)—Proteins and Proteomics 2016; 1864: 1260-1269.
M. K. Herbert, et al., Optimisation of the quantification of glutamine synthetase and myelin basic protein in cerebrospinal fluid by a combined acidification and neutralisation protocol, J. Immunol Methods 381:1-8 (2012).
Y. Horikoshia, et al., Development of Aβ terminal end-specific antibodies and sensitive ELISA for Aβ variant, Biochemical and Biophysical Research Communications, Volume 319, Issue 3, Jul. 2, 2004, 733-737.
J. Ilzecka, Decreased cerebrospinal fluid cGMP levels in patients with amyotrophic lateral sclerosis, 2004 J. Neural Transm 111: 167-72.
K. Johnson-Wood, et al., Amyloid precursor protein processing and A beta42 deposition in a transgenic mouse model of Alzheimer disease, Proc Natl Acad Sci USA. 1997 Feb. 18; 94(4):1550-1555. doi: 10.1073/pnas.94.4.1550.
J. R. Lee, et al., Longitudinal monitoring of antibody responses against tumor cells using magneto-nanosensors with a nanoliter of blood. Nano Lett 17: 6644-6652 (2017).
Q. Li, et al., Improvement of a low pH antigen-antibody dissociation procedure for ELISA measurement of circulating anti-AP antibodies. BMC Neurosci 8, 22 (2007). https://doi.org/10.1186/1471-2202-8-22.
R. M. Lyons, et al., Proteolytic activation of latent transforming growth factor-beta from fibroblast-conditioned medium, 1988 J. Cell Biol 106: 1659-1665.
E. Mandelkow, et al., Structural principles of tau and the paired helical filaments of Alzheimer's disease, Brain Pathol 2007; 17:83-90.
C. M. Mastroianni, et al., Detection of cerebrospinal fluid antibodies against myelin basic protein in patients with AIDS dementia complex, 1991 Mol Cell Neuropathol 14: 227-236.
D. A. McGrowder, et al., Cerebrospinal Fluid Biomarkers of Alzheimer's Disease: Current Evidence and Future Perspectives. Brain Sci 2021; 11:215. doi: 10.3390/brainsci 11020215.
N. Rezaei-Ghaleh, et al., Phosphorylation modifies the molecular stability of 3-amyloid deposits, Nat Commun 2016; Apr. 13; 7:11359.
J. R. Slemmon, et al., Measurement of Aβ1-42 in cerebrospinal fluid is influenced by matrix effects, Journal of Neurochemistry, 2012, 120:325-333.
H. Shahpasand-Kroner, et al., A two-step immunoassay for the simultaneous assessment of Aβ38, Aβ40 and Aβ42 in human blood plasma supports the Aβ42/Aβ40 ratio as a promising biomarker candidate of Alzheimer's disease, Alzheimer's Research & Therapy (2018), 10:121.
H. Vanderstichele, et al., Amino-truncated beta-amyloid42 peptides in cerebrospinal fluid and prediction of progression of mild cognitive impairment, Clin Chem. 2005 September; 51(9):1650-60. doi: 10.1373/clinchem.2005.051201.
H. Vanderstichele, et al., Analytical performance and clinical utility of the INNOTEST PHOSPHO-TAU181P assay for discrimination between Alzheimer's disease and dementia with Lewy bodies. Clin Chem Lab Med 2006; 44:1472-80.
E. Vanmechelen, et al., Quantification of tau phosphorylated at threonine 181 in human cerebrospinal fluid: a sandwich ELISA with a synthetic phosphopeptide for standardization. Neurosci Lett 2000; 285:49-52.
K. G. Warren and I. Catz, A correlation between cerebrospinal fluid mylein basic protein and anti-myelin basic protein in multiple sclerosis patients, 1987 Annal Neurol 21: 183-189.
N. C. Wildburger, et al., Diversity of Amyloid-beta Proteoforms in the Alzheimer's Disease Brain, Sci Rep 2017; 7:9520. doi: 10.1038.
J. Wiltfang, et al. (2001) Elevation of β-Amyloid Peptide 2-42 in Sporadic and Familial Alzheimer's Disease and its Generation in PS1 Knockout Cells. J. Biol Chem 276:42645-42657.
C. Zettler, et al., Detection of increased tissue concentrations of nerve growth factor with an improved extraction procedure, 1996 J. Neurosci Res 46: 581-594.

What is claimed is:

1. A method for determining whether a subject has a condition correlative with a matrix effect comprising (a) admixing (i) a suitably diluted sample of a suitable fluid from the subject and (ii) a suitable amount of a labeled molecule, wherein the labeled molecule is subject to a matrix effect with respect to the suitable fluid in a subject afflicted with the condition, and (b) after a suitable duration under suitable conditions, determining the amount of matrix-unaffected labeled molecule present in the resulting admixture, wherein the subject is afflicted with the condition if the amount of matrix-unaffected labeled molecule determined in step (b) correlates with a positive control for the condition, and wherein the subject is not afflicted with the condition if that amount correlates with a negative control for the condition.

2. The method of claim 1, wherein step (a) further comprises treating the labeled molecule in a manner that facilitates its measurement in step (b).

3. The method of claim 2, wherein treating the labeled molecule in a manner that facilitates its measurement in step (b) comprises contacting it with a suitable amount of an antibody that specifically binds to the labeled molecule.

4. The method of any of claims 1-3, wherein step (a) comprises admixing the suitable amount of labeled molecule with a plurality of serially diluted samples of the fluid.

5. A method for determining whether a subject has a condition correlative with a matrix effect comprising (a) admixing (i) a suitably diluted sample of a suitable fluid from the subject and (ii) suitable amounts of a first labeled molecule and a second labeled molecule, wherein the labeled molecules are subject to a matrix effect with respect to the suitable fluid in a subject afflicted with the condition, and (b) after a suitable duration under suitable conditions, determining the quantitative relationship between matrix-unaffected first labeled molecule and matrix-unaffected second labeled molecule present in the resulting admixture, wherein the subject is afflicted with the condition if the relationship determined in step (b) correlates with a positive control for the condition, and wherein the subject is not afflicted with the condition if that relationship correlates with a negative control for the condition

6. The method of claim 5, wherein step (a) further comprises treating the labeled molecules in a manner that facilitates their measurement in step (b).

7. The method of claim 6, wherein treating the labeled molecules in a manner that facilitates their measurement in step (b) comprises contacting each labeled molecule with a suitable amount of an antibody that specifically binds to it.

8. The method of any of claims 5-7, wherein step (a) comprises admixing the suitable amounts of labeled molecules with a plurality of serially diluted samples of the fluid.

9. The method of any of claims 1-8, wherein the subject is a human.

10. The method of any of claims 1-9, wherein the condition is a condition associated with a neurodegenerative disorder selected from the group consisting of Alzheimer's disease, Parkinson's disease, mild cognitive impairment, and non-Alzheimer's disease dementia.

11. The method of any of claims 1-9, wherein the condition is selected from the group consisting of an amyloidopathy, a tauopathy, neuronal damage, a synucleinopathy, a cerebral proteinopathy, a progranulinopathy, and neuronal inflammation.

12. The method of any of claims 1-11, wherein the suitable fluid is selected from the group consisting of whole blood, serum, plasma, cerebrospinal fluid, saliva, tear fluid, ocular fluid, nasal fluid, vitreous humor, and interstitial fluid.

13. The method of any of claims 1-4 and 9-12, wherein the labeled molecule is selected from the group consisting of a labeled Aβ peptide or a fragment thereof, a labeled tau protein or a fragment thereof, a labeled phosphorylated tau protein or a fragment thereof, labeled progranulin or a fragment thereof, labeled TDP-43 or a fragment thereof, labeled GFAP or a fragment thereof, labeled neurogranin or a fragment thereof, labeled neurofilament or a fragment thereof, and a labeled synuclein or a fragment thereof.

14. The method of any of claims 5-12, wherein the first and second labeled molecules are selected from the group consisting of a labeled Aβ peptide or a fragment thereof, a labeled tau protein or a fragment thereof, a labeled phosphorylated tau protein or a fragment thereof, labeled progranulin or a fragment thereof, labeled TDP-43 or a fragment thereof, labeled GFAP or a fragment thereof, labeled neurogranin or a fragment thereof, labeled neurofilament or a fragment thereof, and a labeled synuclein or a fragment thereof.

15. A kit for use in determining whether a subject has a condition correlative with a matrix effect comprising, in separate compartments, (a) a labeled molecule, wherein the labeled molecule is subject to a matrix effect with respect to a suitable fluid in a subject afflicted with the condition, and (b) an agent useful for treating the labeled molecule to facilitate its measurement in matrix-unaffected form.

16. The kit of claim 15, wherein the kit further comprises, in a separate compartment, an agent useful for measuring the labeled molecule in its matrix-unaffected form.

17. The kit of claim 15 or 16, wherein the labeled molecule is selected from the group consisting of a labeled Aβ peptide or a fragment thereof, a labeled tau protein or a fragment thereof, a labeled phosphorylated tau protein or a fragment thereof, labeled progranulin or a fragment thereof, labeled TDP-43 or a fragment thereof, labeled GFAP or a fragment thereof, labeled neurogranin or a fragment thereof, labeled neurofilament or a fragment thereof, and a labeled synuclein or a fragment thereof.

18. A kit for use in determining whether a subject has a condition correlative with a matrix effect comprising, in separate compartments, (a) a first labeled molecule and a second labeled molecule, wherein each labeled molecule is subject to a matrix effect with respect to a suitable fluid in a subject afflicted with the condition, and (b) an agent useful for treating each of the first and second labeled molecules to facilitate its measurement in matrix-unaffected form.

19. The kit of claim 18, wherein the kit further comprises, in one or more separate compartments, an agent useful for measuring each of the first and second labeled molecules in its matrix-unaffected form.

20. The kit of claim 18 or 19, wherein the first and second labeled molecules are selected from the group consisting of a labeled Aβ peptide or a fragment thereof, a labeled tau protein or a fragment thereof, a labeled phosphorylated tau protein or a fragment thereof, labeled progranulin or a fragment thereof, labeled TDP-43 or a fragment thereof, labeled GFAP or a fragment thereof, labeled neurogranin or a fragment thereof, labeled neurofilament or a fragment thereof, and a labeled synuclein or a fragment thereof.

21. A method for determining whether a human subject has a condition correlative with Alzheimer's disease comprising (a) admixing (i) a suitably diluted sample of a suitable fluid from the subject and (ii) a suitable amount of a labeled Aβ peptide, and (b) after a suitable duration under suitable conditions, determining the amount of matrix-unaffected labeled Aβ peptide present in the resulting admixture, wherein the subject is afflicted with the condition if the amount of matrix-unaffected labeled Aβ peptide determined in step (b) correlates with a positive control for the condition, and wherein the subject is not afflicted with the condition if that amount correlates with a negative control for the condition.

22. The method of claim 21, wherein step (a) further comprises treating the labeled Aβ peptide in a manner that facilitates its measurement in step (b).

23. The method of claim 21 or 22, wherein step (a) comprises admixing the suitable amount of labeled Aβ peptide with a plurality of serially diluted samples of the fluid.

24. The method of any of claims 21-23, wherein the labeled Aβ peptide is selected from the group consisting of labeled Aβ40 peptide or a fragment thereof, labeled Aβ42 peptide or a fragment thereof, labeled Aβ37 peptide or a fragment thereof, labeled Aβ38 peptide or a fragment thereof, labeled Aβ10 peptide or a fragment thereof, labeled Aβ16 peptide or a fragment thereof, and labeled Aβ43 peptide or a fragment thereof.

25. The method of any of claims 21-24, wherein the labeled Aβ peptide is biotinylated Aβ40 peptide or biotinylated Aβ42 peptide.

26. A method for determining whether a human subject has a condition correlative with Alzheimer's disease comprising (a) admixing (i) a suitably diluted sample of a suitable fluid from the subject and (ii) suitable amounts of a first labeled Aβ peptide and a second labeled Aβ peptide, and (b) after a suitable duration under suitable conditions, determining the quantitative relationship between matrix-unaffected first labeled Aβ peptide and matrix-unaffected second labeled Aβ peptide present in the resulting admixture, wherein the subject is afflicted with the condition if the relationship determined in step (b) correlates with a positive control for the condition, and wherein the subject is not afflicted with the condition if that relationship correlates with a negative control for the condition.

27. The method of claim 26, wherein step (a) further comprises treating the labeled Aβ peptides in a manner that facilitates their measurement in step (b).

28. The method of claim 26 or 27, wherein step (a) comprises admixing the suitable amounts of labeled Aβ peptides with a plurality of serially diluted samples of the fluid.

29. The method of any of claims 26-28, wherein each of the labeled Aβ peptides is selected from the group consisting of labeled Aβ40 peptide or a fragment thereof, labeled Aβ42 peptide or a fragment thereof, labeled Aβ37 peptide or a fragment thereof, labeled Aβ38 peptide or a fragment thereof, labeled Aβ10 peptide or a fragment thereof, labeled Aβ16 peptide or a fragment thereof, and labeled Aβ43 peptide or a fragment thereof.

30. The method of any of claims 26-29, wherein the first and second labeled Aβ peptides are biotinylated Aβ40 peptide and biotinylated Aβ42 peptide.

31. The method of any of claims 21-30, wherein the condition is selected from the group consisting of an amyloidopathy, a tauopathy, neuronal damage, a synucleinopathy, a cerebral proteinopathy, a progranulinopathy, and neuronal inflammation.

32. The method of any of claims 21-31, wherein the suitable fluid is selected from the group consisting of whole blood, serum, plasma, cerebrospinal fluid, saliva, tear fluid, ocular fluid, nasal fluid, vitreous humor, and interstitial fluid.

33. A kit comprising, in separate compartments, (a) a labeled Aβ peptide, and (b) an agent useful for treating the labeled Aβ peptide to facilitate the labeled Aβ peptide's measurement in its matrix-unaffected form.

34. The kit of claim 33, wherein the kit further comprises, in a separate compartment, an agent useful for measuring the labeled Aβ peptide in its matrix-unaffected form.

35. The kit of claim 33 or 34, wherein the labeled Aβ peptide is selected from the group consisting of labeled Aβ40 peptide or a fragment thereof, labeled Aβ42 peptide or a fragment thereof, labeled Aβ37 peptide or a fragment thereof, labeled Aβ38 peptide or a fragment thereof, labeled Aβ10 peptide or a fragment thereof, labeled Aβ16 peptide or a fragment thereof, and labeled Aβ43 peptide or a fragment thereof.

36. The kit of any of claims 33-35, wherein the labeled Aβ peptide is biotinylated Aβ40 peptide or biotinylated Aβ42 peptide.

37. A kit comprising, in separate compartments, (a) a first labeled Aβ peptide and a second labeled Aβ peptide, and (b) an agent useful for treating each of the first and second labeled Aβ peptides to facilitate the labeled Aβ peptide's measurement in its matrix-unaffected form.

38. The kit of claim 37, wherein the kit further comprises, in one or more separate compartments, an agent useful for measuring each of the first and second labeled Aβ peptides in its matrix-unaffected form.

39. The method of claim 37 or 38, wherein each of the labeled Aβ peptides is selected from the group consisting of labeled Aβ40 peptide or a fragment thereof, labeled Aβ42 peptide or a fragment thereof, labeled Aβ37 peptide or a fragment thereof, labeled Aβ38 peptide or a fragment thereof, labeled Aβ10 peptide or a fragment thereof, labeled Aβ16 peptide or a fragment thereof, and labeled Aβ43 peptide or a fragment thereof.

40. The kit of any of claims 37-39, wherein the first and second labeled Aβ peptides are biotinylated Aβ40 peptide and biotinylated Aβ42 peptide.
